(19) United States
(12) Patent Application Publication
(10) Pub. No.: US 2005/0258398 A1
(43) Pub. Date: Nov. 24, 2005

(54) Dithia-s-indacene derivative

(75) Inventors: Tohru Kobayashi, Kanagawa-ken (JP); Yoshiki Hasegawa, Kanagawa-ken (JP)

Correspondence Address: SUGHRUE MION, PLLC, 2100 PENNSYLVANIA AVENUE, N.W., SUITE 800, WASHINGTON, DC, 20037, US

(73) Assignee: TAKASAGO INTERNATIONAL CORPORATION

(21) Appl. No.: 11/115,375

(22) Filed: Apr. 27, 2005

(30) Foreign Application Priority Data
May 18, 2004 (JP) JP2004-147116

(51) Int. Cl. C07D 495/02; C09K 19/02; C09K 19/34

(52) U.S. Cl. 252/299.61; 549/43; 349/184

(57) ABSTRACT

A 1,5-dithia-s-indacene or 1,7-dithia-s-indacene derivative represented by general formula (1): 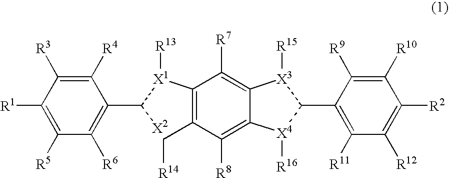
wherein R¹ and R² each independently represents a hydrogen atom, an alkyl group or an alkoxy group; R³-R¹² each independently represents a hydrogen atom, an alkyl group, an alkoxy group, a halogen atom or a cyano group; either of X¹ and X² represents a sulfur atom while the other represents a carbon atom; either of X³ and X⁴ represents a sulfur atom while the other represents a carbon atom; R¹³, R¹⁴, R¹⁵ or R¹⁶ is present only in the case where X¹, X², X³ or X⁴, respectively, is a carbon atom, and R¹³, R¹⁴, R¹⁵ or R¹⁶ each independently represents a hydrogen atom, an alkyl group or a halogen atom; and a broken line independently indicates a double bond in the case where X¹, X², X³ or X⁴ to which the broken line is connected represents a carbon atom or a broken line independently indicates a single bond in case X¹, X², X³ or X⁴ to which the broken line is connected represents a sulfur atom. A liquid crystal composition comprising the 1,5-dithia-s-indacene or 1,7-dithia-s-indacene derivative represented by the general formula (1). An electronic device comprising the liquid crystal composition.

### BACKGROUND OF THE INVENTION

The present invention relates to a liquid crystal compound having a smectic liquid crystal phase and adapted for use as a material for a liquid crystal display or the like.

### DESCRIPTION OF RELATED ART

Compounds with various liquid crystal phases, such as a nematic phase and a smectic phase, are known in the art. Owing to a high regularity in the smectic phase, compounds with a smectic liquid crystal phase are known to be useful for various applications and have been synthesized and reported in various forms. Such liquid crystal compounds are mostly utilized as a mixture of plural liquid crystal compounds and various characteristics are desired for each of such liquid crystal compounds. One desired characteristic is that the liquid crystal phase has a wide temperature range.

### BRIEF SUMMARY OF THE INVENTION

In consideration of the foregoing, an object of the present invention is to provide a novel liquid crystal compound having excellent physical properties.

The present inventors, as a result of intensive investigations for attaining the aforementioned objective, have found that a liquid crystal compound having a dithia-s-indacene structure has a wide temperature range of the liquid crystal phase and has excellent characteristics, and have thus made the present invention.

One embodiment of the present invention is a 1,5-dithia-s-indacene or 1,7-dithia-s-indacene derivative represented by a following general formula (1): 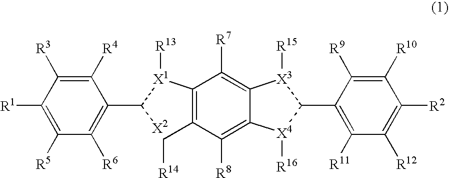
wherein R¹ and R²each independently represents a hydrogen atom, an alkyl group or an alkoxy group; R³, R⁴, R⁵, R⁶, R⁷, R⁸, R⁹, R¹⁰, R¹¹ and R¹² each independently represents a hydrogen atom, an alkyl group, an alkoxy group, a halogen atom or a cyano group; either of X¹ and X² represents a sulfur atom while the other represents a carbon atom; either of X³ and X⁴ represents a sulfur atom while the other represents a carbon atom; R¹³, R¹⁴, R¹⁵ or R¹⁶ is present only in the case where X¹, X², X³ or X⁴, respectively, is a carbon atom, and R¹³, R¹⁴, R¹⁵ or R¹⁶ each independently represents a hydrogen atom, an alkyl group or a halogen atom; and a broken line independently indicates a double bond in the case where X¹, X², X³ or X⁴ to which the broken line is connected represents a carbon atom or a broken line independently indicates a single bond in the case where X¹, X², X³ or X⁴ to which the broken line is connected represents a sulfur atom.

Another embodiment of the present invention is a liquid crystal composition comprising a 1,5-dithia-s-indacene or 1,7-dithia-s-indacene derivative represented by the general formula (1).

Another embodiment of the present invention is an electronic device comprising the liquid crystal composition which comprises a 1,5-dithia-s-indacene or 1,7-dithia-s-indacene derivative represented by the general formula (1).

The 1,5-dithia-s-indacene or 1,7-dithia-s-indacene derivative of the present invention shows a smectic phase in a wide temperature range, and is adapted for use as a material for a liquid crystal display or the like.

### DETAILED DESCRIPTION OF THE INVENTION

In 1,5-dithia-s-indacene and 1,7-dithia-s-indacene derivatives represented by the general formula (1) of the present invention (hereinafter abbreviated collectively as dithiaindacene derivative), the alkyl group represented by R¹ and R² can be a linear or branched alkyl group, for example, with 1-30 carbon atoms, preferably 1-20 carbon atoms. Specific examples of the alkyl group include a methyl group, an ethyl group, an n-propyl group, an isopropyl group, an n-butyl group, an isobutyl group, a sec-butyl group, a tert-butyl group, an n-pentyl group, an n-hexyl group, an n-heptyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, an n-nonadecyl group and an n-eicosyl group.

In the dithiaindacene derivative represented by the general formula (1) of the invention, the alkoxy group represented by R¹ and R² can be a linear of branched alkoxy group, for example, with 1-30 carbon atoms, preferably 1-20 carbon atoms. Specific examples of the alkoxy group include a methoxy group, an ethoxy group, an n-propoxy group, an isopropoxy group, an n-butoxy group, an isobutoxy group, a sec-butoxy group, a tert-butoxy group, an n-pentyloxy group, an n-hexylocyl group, an n-heptyloxy group, an n-octyloxy group, an n-nonyloxy group, an n-decyloxy group, an n-undecyloxy group, an n-dodecyloxy group, an n-tridecyloxy group, an n-tetradecyloxy group, an n-pentadecyloxy group, an n-hexadecyloxy group, an n-heptadecyloxy group, an n-octadecyloxy group, an n-nonadecyloxy group and an n-eicoxyloxy group.

In the dithiaindacene derivative represented by the general formula (1) of the invention, the alkyl group represented by R³-R¹² can be a linear or branched alkyl group, for example, with 1-30 carbon atoms, preferably 1-20 carbon atoms. Thealkoxy group represented by R³-R¹² can be a linear or branched alkoxy group, for example, with 1-30 carbon atoms, preferably 1-20 carbon atoms. Specific examples of the alkyl group and the alkoxy group include those described above for R¹ and R². The halogen atom represented by R¹ and R² can be any halogen atom, preferably a fluorine atom, a chlorine atom or a bromine atom.

In the dithiaindacene derivative represented by the general formula (1) of the invention, the alkyl group represented by R¹³- R¹⁶ can be a linear or branched alkyl group, for example, with 1-30 carbon atoms, preferably 1-20 carbon atoms. The alkoxy group represented by R¹³- R¹⁶ can be a linear or branched alkoxy group, for example, with 1-30 carbon atoms, preferably 1-20 carbon atoms. Specific examples of the alkyl group and the alkoxy group include those described above for R¹ and R². The halogen atom represented by R¹³- R¹⁶ can be any halogen atom, preferably a fluorine atom, a chlorine atom or a bromine atom.

The following Table 1 shows example compounds illustrative of examples of the dithiaindacene derivative of the present invention. The present invention is not limited to such example compounds.

TABLE 1

| Compound No. | Example Compound |
|---|---|
| 1-1 | 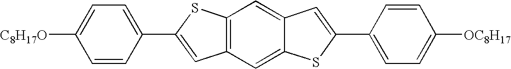 |
| 1-2 | 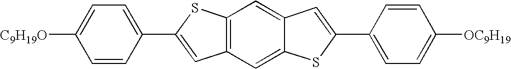 |
| 1-3 | 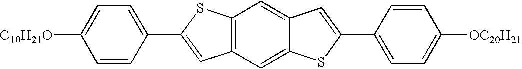 |
| 1-4 | 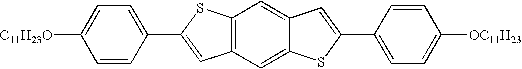 |
| 1-5 | 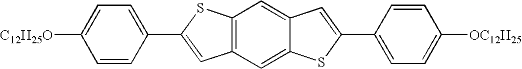 |
| 1-6 | 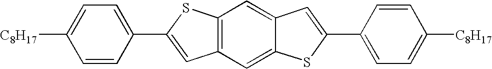 |
| 1-7 | 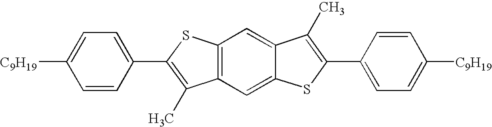 |
| 1-8 | 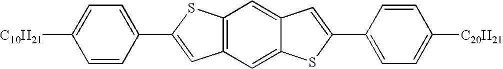 |
| 1-9 | 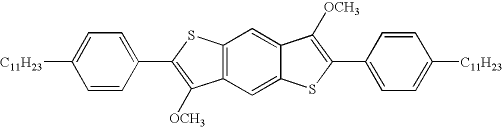 |
| 1-10 | 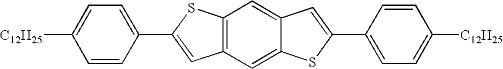 |
| 1-11 | 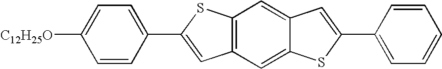 |
| 1-12 | 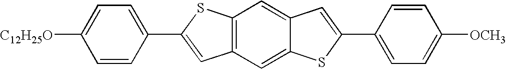 |
| 1-13 | 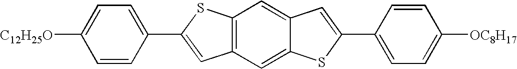 |
| 1-14 | 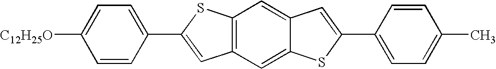 |
| 1-15 | 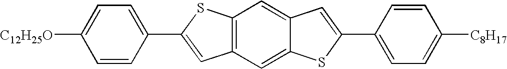 |
| 1-16 | 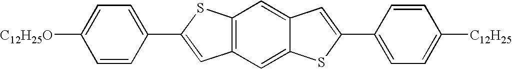 |
| 1-17 | 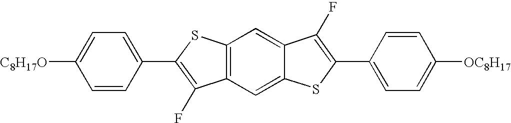 |
| 1-18 | 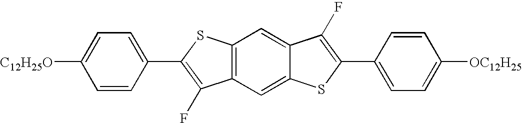 |
| 1-19 | 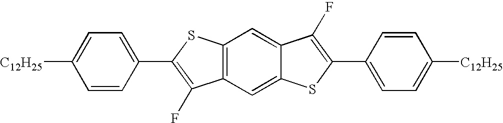 |
| 1-20 | 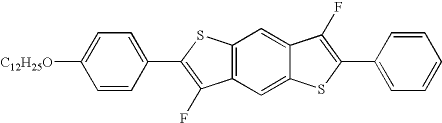 |
| 1-21 | 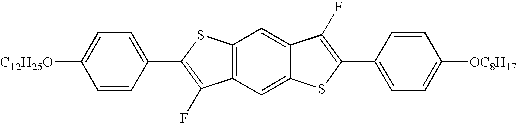 |
| 1-22 | 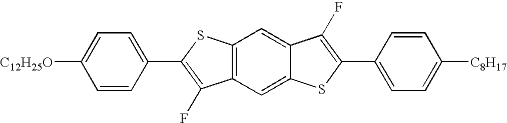 |
| 1-23 | 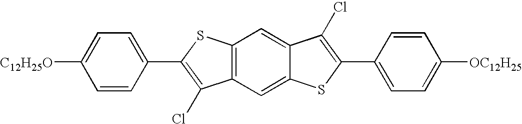 |
| 1-24 | 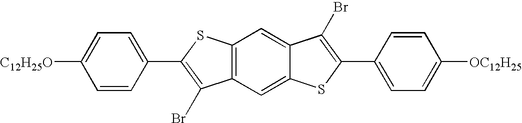 |
| 1-25 | 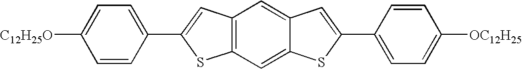 |
| 1-26 | 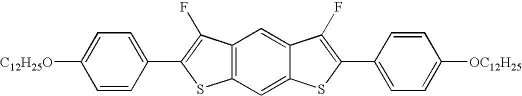 |
| 1-27 | 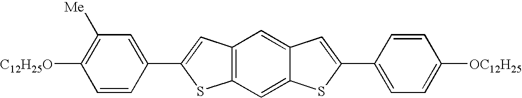 |
| 1-28 | 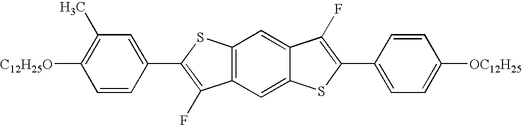 |
| 1-29 | 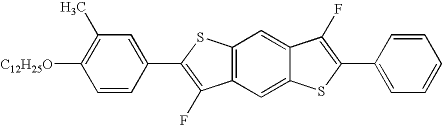 |
| 1-30 | 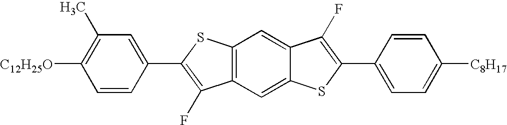 |

The dithiaindacene derivative of the invention represented by the general formula (1) can be synthesized, for example, by a method of the following scheme 1 or a scheme 2, but the synthesizing method of the compound of the invention is not limited to such schemes.

A symmetrical liquid crystal compound can be synthesized by a method of the following scheme 1. An aryl compound (2) and 2-methyl-3-butyn-2-ol are subjected to a coupling reaction and a deprotecting reaction to obtain an acetylene compound (4), which is coupled with a dithiomethyl ether compound (7) in the presence of a palladium catalyst to form a diacetylene compound (8). It is then cyclized with an electrophilic reagent to obtain a compound (9) which, after a metal substitution, is reacted with an electrophilic reagent to obtain a liquid crystal compound (1a). 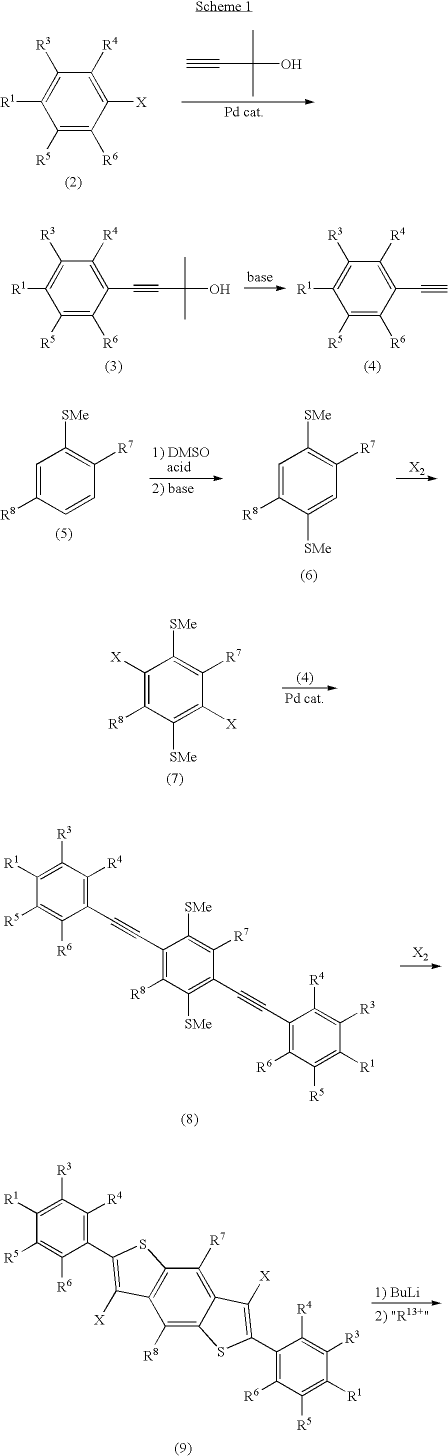 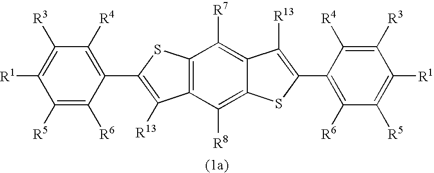
wherein R¹-R⁸ and R¹³ have same meanings as before, and X represents a halogen atom; Pd Cat. represents palladium catalyst; DMSO represents dimethylsulfoxide; and BuLi represents butyllithium.

Also an asymmetrical liquid crystal compound can be synthesized according to the scheme 2. A formyl compound (10), obtained by formylation of a dimethyl dithio ether compound (7), and an acetylene compound (4) are coupled in the presence of a palladium catalyst and subjected to an acetylene formation by a metal benzylsulfone compound to obtain a diacetylene compound (12), which is then subjected to a cyclization reaction and a substitution reaction mentioned above to obtain a liquid crystal compound (1b). 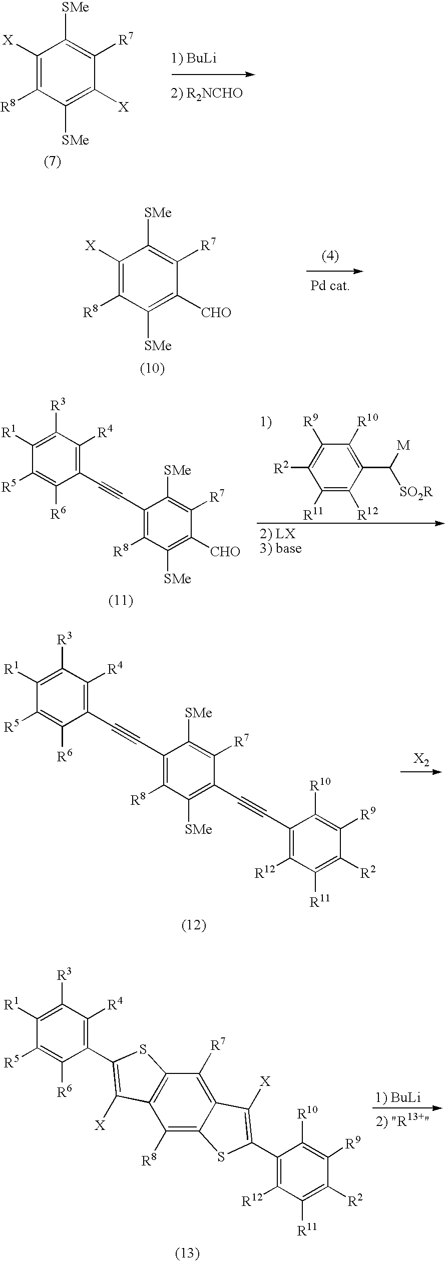 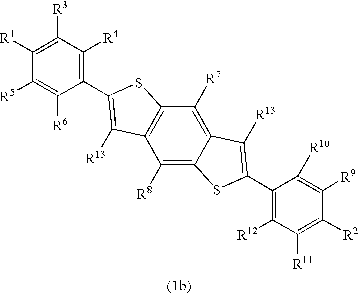
wherein R¹-R¹³ have same meanings as before, X represents a halogen atom Pd Cat. Represents palladium catalyst; LX Represents trimethylsilyl chloride, diethyl phosphoryl chloride, acetic anhydride etc.; and BuLi Represents butyllithium).

The liquid crystal compound of the present invention may be employed in the liquid crystal composition of the present invention singly or as a mixture of two or more liquid crystal compounds.

Also in case of utilizing the liquid crystal compound of the invention as a mixture with another compound, the liquid crystalline property is required to be exhibited only in a mixed state and need not necessarily be exhibited by all the compounds. For example, a composition showing a liquid crystalline property can be prepared by mixing a compound showing a liquid crystalline property and a compound having a core but not showing a liquid crystalline property.

A liquid crystal composition containing the liquid crystal compound of the invention may show any phase that is generally recognized as a liquid crystalline phase in this technical field, but a composition exhibiting a smectic phase is preferable.

The liquid crystal compound or the liquid crystal composition of the invention may be used coated on a substrate, or sealed in a cell prepared with two or more plural substrates, or dropped on a substrate, for example, from a dispenser and then covered with another substrate.

A coating on the substrate may be executed by a method of coating directly a liquid crystal compound or a liquid crystal composition of the invention, or a method of coating and then drying a coating liquid, prepared by dissolving the liquid crystal compound or the liquid crystal composition in a solvent.

A solvent to be employed in the latter method can be an already known solvent. Examples of the solvent include a ketone or a lactone such as acetone, methyl ethyl ketone, cyclohexanone, cyclopentanone, 2-heptanone, methyl isobutyl ketone or γ-butyrolactone; an alcohol such as methanol, ethanol, n-propanol, isopropanol, n-butanol, pentanol, or octanol; an ether such as tert-butyl methyl ether, tetrahydrofuran, dioxane, ethylene glycol monomethyl ether, ethylene glycol monoethyl ether, or ethylene glycol dimethyl ether; an ester such as ethyl acetate, butyl acetate or ethyl propionate; an aromatic compound such as toluene, xylene or chlorobenzene; an amide such as N,N-dimethylacetamide, N,N-dimethylformamide or N-methylpyrrolidone; and a halogenated hydrocarbon such as methylene chloride, chloroform, dichloroethane or trichloroethane. The solvent may be employed singly or in a combination of two or more kinds. A blending proportion of the solvents is suitably adjustable, for example, according to a film thickness required in coating the liquid crystal compound or the liquid crystal composition, a coating condition and the like.

A method of coating can be a known method, such as a wired bar coating, a spin coating, a roll coating, a dip coating, a spray coating, a die coating or an immersion and extraction method. Among these coating methods, a spin coating method and a die coating method are preferable.

A substrate on which the liquid crystal compound or the liquid crystal composition of the invention is to be coated, or a substrate for constituting a cell in which the liquid crystal compound or the liquid crystal composition is to be sealed, can be an organic material or an inorganic material.

An organic material constituting the substrate can be, for example, polyethylene terephthalate, polycarbonate, polyimide, polymethyl methacrylate, polystyrene, polyethylene, polyvinyl chloride, polytetrafluoroethylene, polychlorotrifluoroethylene, polyallylate, polysulfone, or cellulose. Also an inorganic material constituting the substrate can be, for example, silicon, glass or metal.

The surface of the substrate may be treated to form an electrode. A material constituting the electrode can be, for example, indium tin oxide (ITO), indium oxide, zinc oxide, tin oxide, sodium, potassium, magnesium, aluminum, gold, silver, copper, or indium.

A surface of the substrate may be subjected to an aligning treatment for aligning the liquid crystal compound in a predetermined direction. Examples of aligning methods are, for example, a method of rubbing a substrate surface with a cloth, or a method of diagonally evaporating silicon dioxide onto a substrate surface. Also in case of utilizing a substrate not subjected to an aligning process, it is possible to align the liquid crystal compound utilizing an electric field or a magnetic field. Also such aligning methods may be employed singly or in a combination.

In the case where an appropriate aligning property cannot be obtained by rubbing the substrate with a cloth, it is possible, according to known methods, to form an organic thin film such as a polyimide film or a polyvinyl alcohol film on the substrate surface and to rub such thin film with a cloth or the like. It is furthermore possible to utilize a polyimide film providing a pretilt angle as employed in a twisted nematic (TN) cell or a super twisted nematic (STN) cell, or to utilize a photoaligning method instead of the rubbing process.

In the case of controlling the alignment state with an electric field, a substrate having an electrode layer may be employed, and, in such case, an organic film such as the aforementioned polyimide film is preferably formed on the electrode.

The liquid crystal composition of the invention can be utilized for preparing an electronic device such as an EL (electroluminescence) device, a photosensor, an electrophotographic photosensitive member, or an image recording element. An EL device can be prepared by sandwiching a liquid crystal composition of the invention, to which a light emitting material is added if necessary, as a light emitting layer between two electrodes (at least one being a transparent electrode, for example, ITO). Also in a multi-layered organic light emitting device, the liquid crystal material of the invention can be employed as a hole transport layer, an electron transport layer or a light emitting layer. Also in case of a photosensor, the liquid crystal material of the invention, sandwiched between two electrodes (at least one being transparent) can be used for detecting a current change in response to a light irradiation. Also an electrophotographic photosensitive member or an image recording element can be prepared by laminating a charge generation layer and a charge transport layer of the invention on a substrate or an electrode.

A charge generation layer constituting an electrophotographic photosensitive member or an image recording element is formed by an evaporated layer of a charge generation material, or by dispersing a charge generation material in a binder resin.

The charge generation material can be, for example, a polycyclic quinone pigment such as anthanthrone, a perylene pigment, an indigo pigment, such as indigo or thioindigo, a phthalocyanine pigment or a bisazo pigment.

The binder resin can be, for example, a polycarbonate resin, polyvinyl butyral, polystyrene, polyvinyl acetate or an acrylic resin.

A charge generation layer of resin dispersion type can be formed by preparing a dispersion by sufficiently dispersing the aforementioned charge generation material with a binder resin of 3-4 times amount and a solvent, by a suitable method such as a homogenizer, an ultrasonic dispersion, a ball mill, a vibration ball mill, a sand mill, an attriter, a roller mill or a liquid collision type high-speed disperser, and coating and drying such dispersion. A thickness of the charge generation layer is preferably 5 μm or less, particularly preferably 0.1-2 μm.

### EXAMPLES

The present invention will now be illustrated in greater detail by reference to the following examples, but it should be understood that these examples are not to be construed as limiting the scope of the present invention in any way.

A phase transition point was measured with DSC (differential scanning calorimeter) and a polarizing microscope. In relation to the phase transition temperature, Cr means a crystalline phase, SmX means a smectic phase X, SmY means a smectic phase Y, SmZ means a smectic phase Z, Iso means an isotropic liquid, and Dec means a decomposition.

(1) ¹H-NMR: DRX-500 (500 MHz) (manufactured by Bruker Optics Inc.);

GEMINI 2000 (200 MHz) (manufactured by Varian Inc.);

(2) MASS: POLARIS Q (manufactured by Thermo Electron Corp.);

(3) Phase transition temperature: DSC-600 (manufactured by Shimadzu Corp.).

### Example 1

### Synthesis of Example Compound 1-1

### (1) Synthesis of 1-bromo-4-octyloxybenzene (2a)

In a nitrogen atmosphere, 19.1 g (476.9 mmol) of sodium hydride (60 wt. %) was suspended in 400 mL of N,N-dimethylformamide (DMF), and, under cooling with ice, a solution of 75.0 g (433.5 mmol) of 4-bromophenol in 100 mL of DMF was dropwise added. After the addition, the mixture was warmed up to the room temperature, then agitated for 1 hour, added with 100.5 g (520.2 mmol) of octyl bromide and agitated for 2 hours. Then after the reaction was terminated by adding water, the reaction mixture was extracted with toluene and the organic phase was washed with water and concentrated. A residue was purified by a distillation under a reduced pressure (150-152° C./133.3 Pa) to obtain the above-mentioned compound (2a) in an amount of 89.5 g (313.7 mmol, 72.4%).

¹H-NMR (200 MHz, CDCl₃) δ: 0.85-0.92 (m, 3H), 1.26-1.47 (m, 10H), 1.73-1.80 (m, 2H), 3.91 (t, J=6.6 Hz, 2H), 6.77 (d, J=8.6 Hz, 2H), 7.36 (d, J=8.6 Hz, 2H);

MS (m/z): 286.1, 172.1.

### (2) Synthesis of 4-(4-octyloxyphenyl)-2-methyl-3-butyn-2-ol (3a)

In a nitrogen atmosphere, 25.1 g (88.0 mmol) of 1-bromo-4-octyloxybenzene (2a), 13.3 g (158.4 mmol) of 2-methyl-3-butyn-2-ol, 156.0 mg (0.88 mmol) of palladium chloride and 335.2 mg (1.76 mmol) of CuI were dissolved in 125 mL of tetrahydrofuran, then 26.7 g (264.0 mmol) of diisopropylamine and 0.75 mL (1.85 mmol) of tri-tert-butylphosphine [2.48 mol/L toluene solution] was dropwise added and the mixture was agitated at 70° C. overnight. After an addition of water, an extract with toluene was washed with a saturated aqueous solution of ammonium chloride, further washed twice with water and concentrated. A residue was purified by a column chromatography to obtain the above-mentioned compound (3a) in an amount of 22.1 g (76.6 mmol, 87.1%)

¹H-NMR (200 MHz, CDCl₃) δ: 0.85-0.92 (m, 3H), 1.21-1. 43 (m, 10H), 1.60 (s, 6H), 1.70-1.81 (m, 2H), 2.00 (s, 1H), 3.94 (t, J=6.6 Hz, 2H), 6.82 (d, J=8.8 Hz, 2H), 7.34 (d, J=8.8 Hz, 2H)

MS m/z: 288.2, 273.3, 161.2.

### (3) Synthesis of 1-ethynyl-4-octyloxybenzene (4a)

A mixture of 33.2 g (115.1 mmol) of 4-(4-octyloxyphenyl)-2-methyl-3-butyn-2-ol (3a), 4.6 g (115.1 mmol) of sodium hydroxide and 165 mL of toluene was agitated for 1 hour under heating and under elimination of toluene with a Dean-Stark trap. After cooling, solid was filtered off and the filtrate was washed with a 10% aqueous solution of sulfuric acid and with water, then concentrated and a residue was purified by a column chromatography to obtain the above-mentioned compound (4a) in an amount of 24.2 g (104.9 mmol, 93.9%).

1H-NMR (200 MHz, CDCl₃) δ: 0.85-0.92 (m, 3H), 1.30-1.48 (m, 10H), 1.71-1.81 (m, 2H), 2.98 (s, 1H), 3.95 (t, J=6.4 Hz, 2H), 6.82 (d, J=8.7 Hz, 2H), 7.41 (d, J=8.7 Hz, 2H);

MS m/z: 230.2, 118.2.

### (4) Synthesis of 1,4-dimethylthiobenzene (6a)

In a nitrogen atmosphere, 51.4 g (413.8 mmol) of thioanisole (5a) was added with 32.3 g (413.8 mmol) of dimethylsulfoxide and ice cooled, and 99.4 g (662.1 mmol) of trifluoromethanesulfonic acid were dropwise added. After the addition, the mixture was warmed up the room temperature, then agitated for 4 hours, and added with diethyl ether and precipitating crystals were separated by filtration. The crystals were washed with diethyl ether to obtain 100.4 g of dimethyl{4-(methylthio)phenyl}sulfonium triflate. The product was then dissolved in 327.3 g (4.14 mol) of pyridine and refluxed for 2 hours. The reaction was terminated by adding 10% aqueous solution of sulfuric acid, and, after an extraction with toluene, the organic phase was washed with water. It was then concentrated to dry to obtain the above-mentioned compund (6a) in an amount of 41.0 g (240.8 mmol, 58.2 5 in 2 steps).

1H-NMR (200 MHz, CDCl₃) δ: 2.46 (s, 6H), 7.20 (s, 4H);

MS m/z: 170.1, 155.1.

### (5) Synthesis of 2,5-dibromo-1,4-dimethylthiobenzene (7a)

In a nitrogen atmosphere, 5.96 g (35.0 mmol) of 1,4-dimethylthiobenzene (6a) was dissolved in 120 mL of dichloromethane and sealed from light. Under cooling with ice, 533.0 mg (2.10 mmol) of iodine was added and the mixture was agitated for 30 minutes. Then 28.0 g (175.0 mmol) of bromine was dropwise added under cooling with ice, and the mixture was warmed up to the room temperature and agitated for 3 days. The reaction was terminated by adding a 0.5% aqueous solution of sodium hydrogensulfite, then the mixture was extracted with chloroform and the organic phase was washed three times with water. After a concentration step, a residue was recrystallized from chloroform to obtain the above-mentioned compound (7a) in an amount of 8.54 g (26.0 mmol, 74.4%).

¹H-NMR (200 MHz, acetone-d6) δ: 2.56 (s, 6H), 7.40 (s, 2H);

MS m/z: 327.9, 321.9, 234.0.

### (6) Synthesis of 2,5-di(4-octyloxyphenylethynyl)-1,4-dimethylthiobenzene 8a)

In a nitrogen atmosphere, 1.80 g (5.49 mmol) of 2,5-dibromo-1,4-dimethylthiobenzene (7a), 123.2 mg (0.549 mmol) of palladium acetate, 719.5 mg (2.74 mmol) of triphenylphosphine and 209.0 mg (1.10 mmol) of copper (I) iodide were dissolved in 100 mL of THF, then 2.22 g (21.9 mmol) of triethylamine and 3.03 g (13.2 mmol) of 1-ethynyl-4-octyloxybenzene (4a) were added, and the mixture was heated under agitation for 15 hours at 70° C. The reaction was terminated by adding water, then the mixture was extracted with toluene, and the organic phase was washed with a saturated aqueous solution of ammonium chloride and with water, and concentrated. A residue was purified with a column chromatography and recrystallized from hexane to obtain the above-mentioned compound (8a) in an amount of 1.91 g (3.05 mmol, 55.5%).

¹H-NMR (200 MHz, CDCl₃) δ: 0.86-0.89 (m, 6H), 1.31-1.45 (m, 20H), 1.75-1.83 (m, 4H), 2.52 (s, 6H), 3.97 (t, J=6.50 Hz, 4H), 6.88 (d, J=8.8 Hz, 4H), 7.51 (d, J=8.8 Hz, 4H)

MS m/z: 626.4, 513.3, 401.2.

### (7) Synthesis of 3,7-diiodo-2,6-bis(4-octyloxyphenyl)-1,5-dithia-s-indacen e (9a)

In a nitrogen atmosphere, 1.91 g (3.04 mmol) of 2,5-di(4-octyloxyphenylethynyl)-1,4-dimethylthiobenzene (8a) was dissolved in 40 mL of dichloromethane, and a solution of 3.09 g (12.2 mmol) of iodine in 250 mL of dichloromethane was added at the room temperature. After agitation for 30 minutes, an aqueous solution of sodium hydrogensulfite was added, then the organic phase was washed four times with water, and filtered after adding acetone, and the resulting crystals were washed with acetone to obtain the above-mentioned compound (9a) in an amount of 2.25 g (2.64 mmol, 86.8%).

¹H-NMR (200 MHz, CDCl₃) δ: 0.87-0.90 (m, 6H), 1.22-1.42 (m, 20H), 1.80-1.83 (m, 4H), 4.04 (t, J=6.50 Hz, 4H), 7.02 (d, J=8.80 Hz, 4H), 7.68 (d, J=8.80 Hz, 4H), 8.22 (s, 2H);

MS m/z: 849.6, 737.6, 625.6, 371.9.

### (8) Synthesis of Example Compound 1-1

In a nitrogen atmosphere, 2.53 g (2.97 mmol) of 3,7-diiodo-2,6-bis(4-octyloxyphenyl)-1,5-dithia-s-indacene (9a) was dissolved in 100 mL of THF. 7.48 mL (11.9 mmol) of n-butyl lithium [1.59 mol/L, n-hexane solution] was dropwise added under cooling with ice, and the mixture was agitated for 1 hour. After the reaction was terminated with a 10% aqueous solution of sulfuric acid, the mixture was extracted with hexane, then the organic phase was washed with water and the solvent was distilled off. A recrystallization from DMF provided the above-mentioned compound (1-1) in an amount of 490.0 mg (0.819 mmol, 27.5%).

¹H-NMR (500 MHz, DMF-d₇, 130° C.) δ: 0.95 (t, J=7.1 Hz, 6H), 1.36-1.47 (m, 10H), 1.54-1.58 (m, 4H), 1.85-1.89 (m, 4H), 4.16 (t, J=6.6 Hz, 4H), 7.11 (d, J=8.7 Hz, 4H), 6.88 (s, 2H), 7.77 (d, J=8.7 Hz, 4H), 8.34 (s, 2H);

MS m/z: 598.2, 486.1, 374.1;

DSC: Cr 132.6° C., SmX 153.6° C., SmY 360.4° C., Dec.

### Example 2

### Synthesis of Example Compound 1-5

### (1) Synthesis of 1-bromo-4-dodecyloxybenzene (2b)

In a nitrogen atmosphere, 19.1 g (476.9 mmol) of sodium hydride (60 wt. %) was suspended in 400 mL of N,N-dimethylformamide (DMF), and, under cooling with ice, a solution of 75.0 g (433.5 mmol) of 4-bromophenol in 100 mL of DMF was dropwise added. After the addition, the mixture was warmed up to the room temperature, then agitated for 1 hour, added with 124.2 g (498.5 mmol) of dodecyl bromide and agitated for 2 hours. Then after the reaction was terminated by adding water, the reaction mixture was extracted with toluene and the organic phase was washed with water and concentrated. A residue was purified by a distillation under a reduced pressure (173° C./66.7 Pa) to obtain the above-mentioned compound (2b) in an amount of 105.9 (310.4 mmol, 71.6%).

¹H-NMR (200 MHz, CDCl₃) δ: 0.85-0.91 (m, 3H), 1.26-1.53 (m, 18H), 1.69-1.80 (m, 2H), 3.91 (t, J=6.6 Hz, 2H), 6.76 (d, J=7.0 Hz, 2H), 7.36 (d, J=7.0 Hz, 2H);

MS (m/z): 341.3, 172.1.

### (2) Synthesis of 4-(4-dodecyloxyphenyl)-2-methyl-3-butyn-2-ol (3b)

In a nitrogen atmosphere, 34.8 g (89.6 mmol) of 1-iodo-4-dodecyloxybenzene, 13.6 g (161.3 mmol) of 2-methyl-3-butyn-2-ol, 402.4 mg (1.79 mmol) of palladium chloride, 2.35 g (8.96 mmol) of triphenylphosphine, 682.7 mg (3.58 mmol) of CuI and 27.2 g (268.8 mmol) of triethylamine were dissolved in 175 mL of THF, and the mixture was agitated overnight at 70° C. After an addition of water, an extract with toluene was washed with a saturated aqueous solution of ammonium chloride, further washed twice with water and concentrated. A residue was purified by a column chromatography to obtain the above-mentioned compound (3b) in an amount of 21.0 g (61.0 mmol, 68.0%).

¹H-NMR (200 MHz, CDCl₃) δ: 0.85-0.96 (m, 3H), 1.21-1.47 (m, 18H), 1.61 (s, 6H), 1.74-1.80 (m, 2H), 1.98 (s, 1H), 3.94 (t, J=6.5 Hz, 2H), 6.81 (d, J=8.8 Hz, 2H), 7.34 (d, J=8.8 Hz, 2H);

MS m/z: 344.2, 329.3, 161.2.

### (3) Synthesis of 1-ethynyl-4-dodecyloxybenzene (4b)

105 mL of toluene was added to 21.0 g (61.0 mmol) of 4-(4-dodecyloxyphenyl)-2-methyl-3-butyn-2-ol and 2.44 g (61.0 mmol) of sodium hydroxide and the mixture was agitated for 1 hour under heating and under elimination of toluene with a Dean-Stark trap. After cooling, solid was filtered off and the filtrate was washed with a 10% aqueous solution of sulfuric acid and with water, then concentrated and a residue was purified by a column chromatography to obtain the above-mentioned compound in an amount of 13.5 g (47.1 mmol, 77.3%)

1H-NMR (200 MHz, CDCl₃) δ: 0.85-0.92 (m, 3H), 1.30-1.48 (m, 18H), 1.71-1.81 (m, 2H), 2.99 (s, 1H), 3.95 (t, J=6.4 Hz, 2H), 6.82 (d, J=8.8 Hz, 2H), 7.41 (d, J=8.8 Hz, 2H);

MS m/z: 286.3, 147.2, 133.2.

### (4) Synthesis of 2,5-di(4-dodecyloxyphenylethynyl)-1,4-dimethylthiobenzene (8b)

In a nitrogen atmosphere, 3.00 g (9.14 mmol) of 2,5-dibromo-1,4-dimethylthiobenzene (7a), 205.3 mg (0.914 mmol) of palladium acetate, 1.20 g (4.57 mmol) of triphenylphosphine and 348.3 mg (1.83 mmol) of copper (I) iodide were dissolved in 100 mL of THF, then 3.70 g (36.6 mmol) of triethylamine and 6.29 g (21.9 mmol) of 1-ethynyl-4-dodecyloxybenzene (5b) were added, and the mixture was heated under agitation for 15 hours at 70° C. The reaction was terminated by adding water, then the mixture was extracted with toluene, and the organic phase was washed with a saturated aqueous solution of ammonium chloride and with water, and concentrated. A residue was purified with a column chromatography and recrystallized from hexane to obtain the above-mentioned compound (8b) in an amount of 4.22 g (5.71 mmol, 62.4%)

¹H-NMR (200 MHz, CDCl₃) δ: 0.85-0.91 (m, 6H), 1.27-1.56 (m, 36H), 1.75-1.78 (m, 4H), 3.92-4.01 (t, J=6.4 Hz, 4H), 6.87 (d, J=8.8 Hz, 4H), 7.34 (s, 2H), 7.51 (d, J=8.8 Hz, 4H);

MS m/z: 738.2, 570.2, 402.1.

### (5) Synthesis of 3,7-diiodo-2,6-bis(4-dodecyloxyphenyl)-1,5-dithia-s-indacene (9b)

In a nitrogen atmosphere, 4.22 g (5.71 mmol) of 2,5-di(4-dodecyloxyphenylethynyl)-1,4-dimethylthiobenzene (8b) was dissolved in 40 mL of dichloromethane, and a solution of 5.80 g (22.8 mmol) of iodine in 250 mL of dichloromethane was added at the room temperature. After agitation for 30 minutes, an aqueous solution of sodium hydrogensulfite was added until the color disappeared, then the organic phase was washed four times with water, and filtered after adding acetone, and the resulting crystals were washed with acetone to obtain the above-mentioned compound (9b) in an amount of 3.20 g (3.32 mmol, 58.2%)

¹H-NMR (200 MHz, CDCl₃) δ: 0.81-0.95 (m, 6H), 1.28-1.42 (m, 36H), 1.79-1.93 (m, 4H), 4.04 (t, J=6.7 Hz, 4H), 7.02 (d, J=8.2 Hz, 4H), 7.68 (d, J=8.2 Hz, 4H), 8.22 (s, 2H);

MS m/z: 962.1, 794.0, 625.9.

### (6) Synthesis of Example Compound 1-5

In a nitrogen atmosphere, 3.20 g (3.32 mmol) of 3,7-diiodo-2,6-bis(4-dodecyloxyphenyl)-1,5-dithia-s-indacene (9b) was suspended in 100 mL of THF. Then, after cooling to −78° C., 8.36 mL (13.3 mmol) of n-butyl lithium [1.59 mol/L, n-hexane solution] was dropwise added, and the mixture was agitated for 1 hour. After the reaction was terminated with a 10% aqueous solution of sulfuric acid, the mixture was extracted with hexane, then the organic phase was washed with water and the solvent was distilled off. A recrystallization of the resultant from N-methyl-2-pyrrolidone provided the above-mentioned compound (1-5) in an amount of 1.59 g (2.28 mmol, 68.7%).

¹H-NMR (500 MHz, DMF-d₇, 130° C.) δ: 0.88 (t, J=7.10 Hz, 16H), 1.32-1.43 (m, 32H), 1.50-1.51 (m, 4H), 1.80-1.82 (m, 4H), 4.11 (t, J=6.60 Hz, 4H), 7.06 (d, J=8.70 Hz, 4H), 7.64 (s, 2H), 7.72 (d, J=8.70 Hz, 4H), 8.29 (s, 2H);

MS m/z: 710.3, 542.2, 374.1;

DSC: Cr 127.0° C., SmX 371.2° C., Iso.

### Example 3

### Synthesis of Example Compound 1-17

### (1) Synthesis of Example Compound 1-17

In a nitrogen atmosphere, 1.27 g (1.49 mmol) of 3,7-diiodo-2,6-bis(4-octyloxyphenyl)-1,5-dithia-s-indacene (9a) was suspended in 100 mL of THF. Then, after cooling to −78° C., 3.76 mL (5.97 mmol) of n-butyl lithium [1.59 mol/L, n-hexane solution] was dropwise added, and the mixture was agitated for 1 hour. Then, at −78° C., a solution of 1.88 g (5.97 mmol) of N-fluorobenzenesulfonimide in 20 mL of THF was dropwise added, and the mixture was agitated for 2 hours. After the reaction was terminated by adding water, the mixture was extracted with toluene, then the organic phase was washed with water and the solvent was distilled off. After a recrystallization from N-methyl-2-pyrrolidone, the crystals were washed with water and further recrystallized from N-methyl-2-pyrrolidone to obtain the above-mentioned compound (1-17) in an amount of 136.2 mg (0.21 mmol, 14.4%)

¹H-NMR (500 MHz, DMF-d₇, 130° C.) δ: 0.89-0.91 (m, 6H), 1.34-1.42 (m, 16H), 1.52-1.53 (m, 4H), 1.82-1.82 (m, 4H), 4.13 (t, J=6.4 Hz, 4H), 7.12 (d, J=7.9 Hz, 4H), 7.74 (d, J=7.9 Hz, 4H), 8.28 (s, 2H);

MS m/z: 634.2, 616.2, 410.0;

DSC: Cr 136.2° C., SmX 136.2° C., SmY 212.4° C., SmX 247.2° C., SmZ 354.6° C., Iso.

While the invention has been described in detail and with reference to specific embodiments thereof, it will be apparent to one skilled in the art that various changes and modifications can be made therein without departing from the spirit and scope thereof.

1. A 1,5-dithia-s-indacene or 1,7-dithia-s-indacene derivative represented by general formula (1): 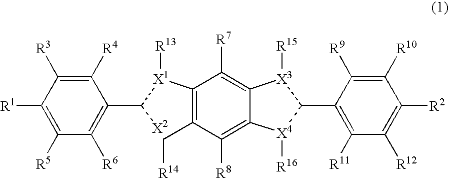
wherein R¹ and R²each independently represents a hydrogen atom, an alkyl group or an alkoxy group; R³, R⁴, R⁵, R⁶, R⁷, R⁸, R⁹, R¹⁰, R¹¹ and R¹² each independently represents a hydrogen atom, an alkyl group, an alkoxy group, a halogen atom or a cyano group; either of X¹ and X² represents a sulfur atom while the other represents a carbon atom; either of X³ and X⁴ represents a sulfur atom while the other represents a carbon atom; R¹³, R¹⁴, R¹⁵ or R¹⁶ is present only in the case where X¹, X², X³ or X⁴, respectively, is a carbon atom, and R¹³, R¹⁴, R¹⁵ and R¹⁶ each independently represents a hydrogen atom, an alkyl group or a halogen atom; and a broken line independently indicates a double bond in the case where X¹, X², X³ or X⁴ to which the broken line is connected represents a carbon atom or a broken line independently indicates a single bond in the case where X¹, X², X³ or X⁴ to which the broken line is connected represents a sulfur atom.

2. The 1,5-dithia-s-indacene or 1,7-dithia-s-indacene derivative of claim 1, which is a 1,5-dithia-s-indacene derivative.

3. The 1,5-dithia-s-indacene or 1,7-dithia-s-indacene derivative of claim 1, which is a 1,7-dithia-s-indacene derivative.

4. The 1,5-dithia-s-indacene or 1,7-dithia-s-indacene derivative of claim 1, wherein R¹³, R¹⁴, R¹⁵ and R¹⁶, when present, are independently selected from the group consisting of hydrogen, fluorine, chlorine, —OCH₃ and —CH₃.

5. The 1,5-dithia-s-indacene or 1,7-dithia-s-indacene derivative of claim 1, wherein R¹ and R² both have the same structure.

6. The 1,5-dithia-s-indacene or 1,7-dithia-s-indacene derivative of claim 1, wherein R¹ and R² do not have the same structure.

7. The 1,5-dithia-s-indacene or 1,7-dithia-s-indacene derivative of claim 1, wherein R¹ and R² are independently alkoxy.

8. The 1,5-dithia-s-indacene or 1,7-dithia-s-indacene derivative of claim 1, wherein R¹ and R² are independently alkyl.

9. The 1,5-dithia-s-indacene or 1,7-dithia-s-indacene derivative of claim 1, wherein said 1,5-dithia-s-indacene or 1,7-dithia-s-indacene derivative is selected from the group consisting of structural formula I-1-I-30:

| Compound No. | |
|---|---|
| 1-1 | 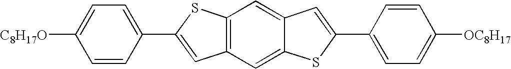 |
| 1-2 | 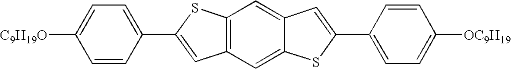 |
| 1-3 | 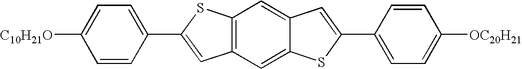 |
| 1-4 | 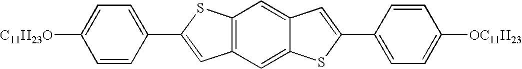 |
| 1-5 | 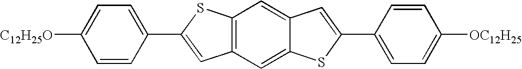 |
| 1-6 | 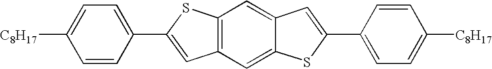 |
| 1-7 | 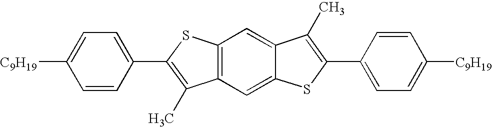 |
| 1-8 | 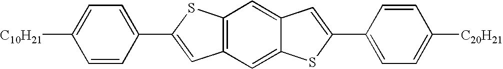 |
| 1-9 | 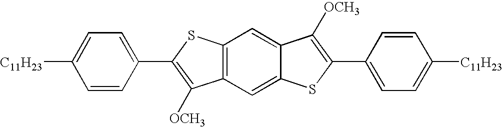 |
| 1-10 | 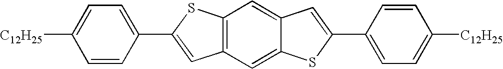 |
| 1-11 | 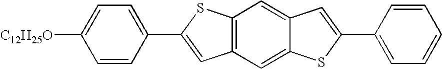 |
| 1-12 | 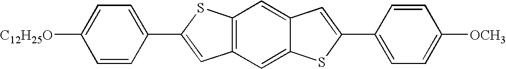 |
| 1-13 | 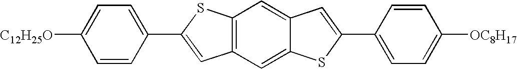 |
| 1-14 | 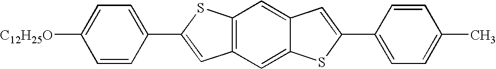 |
| 1-15 | 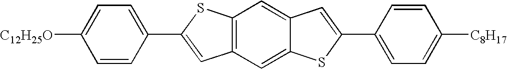 |
| 1-16 | 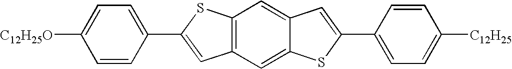 |
| 1-17 | 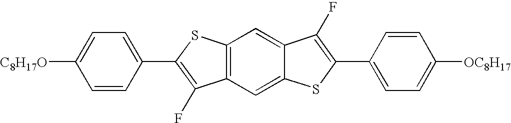 |
| 1-18 | 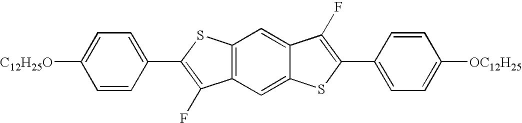 |
| 1-19 | 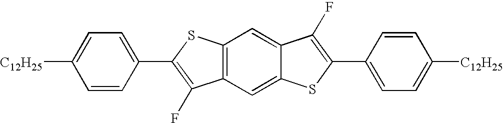 |
| 1-20 | 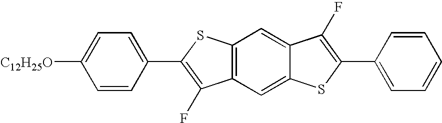 |
| 1-21 | 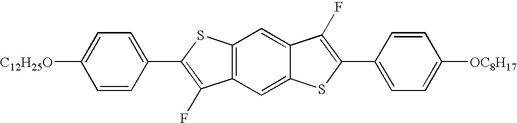 |
| 1-22 | 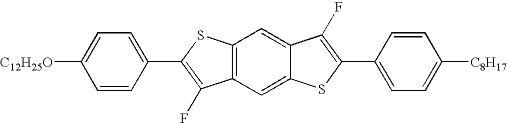 |
| 1-23 | 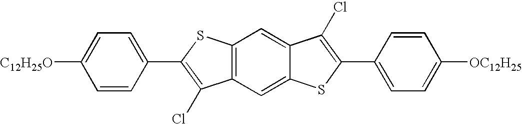 |
| 1-24 | 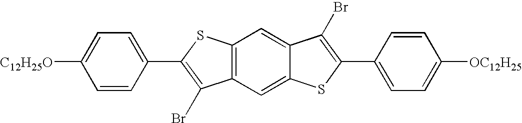 |
| 1-25 | 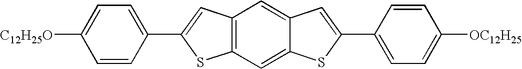 |
| 1-26 | 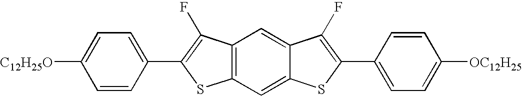 |
| 1-27 | 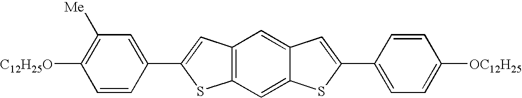 |
| 1-28 | 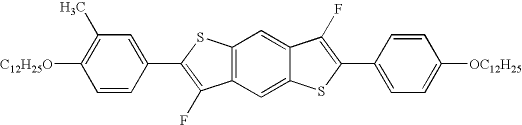 |
| 1-29 | 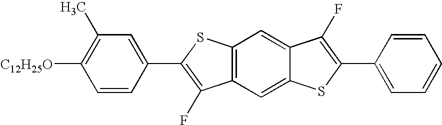 |
| 1-30 | 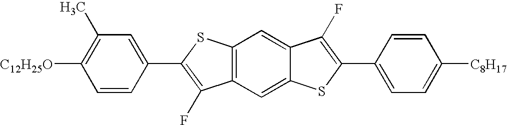 |

10. The 1,5-dithia-s-indacene or 1,7-dithia-s-indacene derivative of claim 9, wherein said 1,5-dithia-s-indacene or 1,7-dithia-s-indacene derivative is selected from the group consisting of structural formula I-1, I-5 and I-17.

11. A liquid crystal composition comprising at least one 1,5-dithia-s-indacene or 1,7-dithia-s-indacene derivative according to claim 1.

12. The liquid crystal composition of claim 11, where said composition exhibits a smectic phase.
